(19) United States
(12) Patent Application Publication
(10) Pub. No.: US 2022/0287842 A1
(43) Pub. Date: Sep. 15, 2022

(54) PERCUTANEOUS MINIMALLY INVASIVE POSTERIOR VENTRICULAR ARTIFICIAL CHORDAE DEVICES AND SYSTEMS AND METHODS FOR REPAIR OF MITRAL REGURGITATION IN A BEATING HEART

(71) Applicant: THE BOARD OF TRUSTEES OF THE LELAND STANFORD JUNIOR UNIVERSITY, Stanford, CA (US)

(72) Inventors: Y. Joseph Woo, Stanford, CA (US); Yuanjia Zhu, Stanford, CA (US); Annabel M. Imbrie-Moore, Stanford, CA (US); Matthew Park, Piedmont, CA (US); Michael John Paulsen, Los Altos, CA (US); John W. MacArthur, JR., Stanford, CA (US)

(21) Appl. No.: 17/830,247

(22) Filed: Jun. 1, 2022

Related U.S. Application Data
(63) Continuation of application No. PCT/US2020/063832, filed on Dec. 8, 2020.
(60) Provisional application No. 62/945,199, filed on Dec. 8, 2019.

(51) Int. Cl. A61F 2/24

(52) U.S. Cl. CPC A61F 2/2466; A61F 2/2457; A61B 2017/00243

(57) ABSTRACT

Devices, systems, and methods are provided for mitral repair of a mitral valve within a heart of a subject that includes introducing a distal portion of a delivery device carrying an artificial chordae into the vasculature of the subject; advancing the distal portion into a right atrium of the heart and trans-septally from the right atrium into a left atrium of the heart; directing the distal portion through a mitral valve of the heart into a left ventricle of the heart; securing a distal end of the artificial chordae to a posterior ventricular wall of the heart adjacent the mitral valve; coupling a leaflet anchor end of the artificial chordae to a posterior leaflet of the mitral valve; adjusting a distance between the leaflet anchor and the distal end of the artificial chordae until coaptation of the mitral valve is improved and/or regurgitation is eliminated; and removing the delivery device from the subject's body.

### RELATED APPLICATION DATA

The present application is a continuation of co-pending International Application No. PCT/US2020/063832, filed Dec. 8, 2020, which claims benefit of co-pending U.S. provisional patent application Ser. No. 62/945,199, filed Dec. 8, 2019, the entire disclosures of which are expressly incorporated by reference herein.

### TECHNICAL FIELD

The present invention relates to devices and methods for repairing mitral valve regurgitation, and, more particularly, to devices, systems, and methods for percutaneously implanting artificial chordae for repairing mitral valve regurgitation in a beating heart.

### BACKGROUND

Valvular heart disease is a common cause of morbidity and mortality, with 2.5% of the U.S. population afflicted. Five and eight year survival rates for those with valvular heart disease is 79% and 68%, respectively, rivaling many types of cancer. Degenerative mitral valve regurgitation is the most common type of valve disease. Of the nearly 2% of the U.S. population with mitral regurgitation, nearly 10% will progress to develop severe enough regurgitation to necessitate surgical intervention.

Studies have demonstrated that mitral valve repair is preferred over valve replacement, as repair improves survival, preserves left ventricular function, and results in greater freedom from reoperation as structural valve deterioration is not a critical issue with repair. However, repair today often requires open heart surgery, which is obviously higher risk than many of the percutaneous devices available today.

Therefore, new devices and methods that facilitate mitral valve repair would be useful.

### SUMMARY

The present invention is directed to devices and methods for repairing mitral valve regurgitation, and, more particularly, to devices, systems, and methods for percutaneously implanting artificial chordae for repairing mitral valve regurgitation, e.g., in a beating heart.

The treatment of aortic stenosis has been revolutionized by the development of transcutaneous aortic valve replacement (TAVR), which is now the preferred procedure. The same revolution is occurring with the treatment of mitral disease; however, as mentioned above, mitral replacement is suboptimal compared to repair. For this reason, the development of percutaneous mitral repair technologies is paramount to furthering this technology and may become a multi-billion-dollar industry.

Current repair devices for the mitral valve are suboptimal and include clips and artificial chordae anchored at the heart's apex. However, apical anchoring may result in excessively high failure rates due to higher forces and loading on longer chordae.

What is proposed herein is a percutaneous manifestation of an operation, namely percutaneous posterior ventricular anchoring chordal (“PPVAN”) repair, whereby an artificial chordae is anchored posteriorly in the ventricular muscle behind the posterior leaflet. The resulting artificial chordae may be relatively short, and the forces experienced by this repair structure may be extremely small, with a low resulting failure rate. Because of the design of the operation, it is possible and entirely feasible to perform this repair percutaneously without the need for surgery and while the heart remains beating normally.

In accordance with an exemplary embodiment, a percutaneous posterior ventricular artificial chordae repair device and a trans-septal delivery system are provided. During use, a patient's femoral vein may be accessed, e.g., using the Seldinger technique, and a guidewire may be passed through the patient's vasculature into the right atrium of the patient's heart. Next, the atrial septum may be perforated, and a steerable catheter may be navigated through the atrial septum into the left atrium of the heart. Next, the artificial chordae device may be navigated through the catheter into the left atrium.

In an exemplary embodiment, the artificial chordae device includes three primary components, namely an anchoring screw, barb, or other tissue anchor, which is rigidly fixed to the distal end of a polytetrafluoroethylene (PTFE) or other filament (artificial chordae), which then passes through a leaflet anchor. The distance between the tissue anchor and leaflet anchor approximates the length of the artificial chordae; this length may be set based on patient imaging prior to the operation. In one embodiment, the leaflet anchor is movable on the filament and is configured to attach the filament to a valve leaflet, which allows for the length of the artificial chordae to be adjusted prior to final securing to adjust the leaflet coaptation for optimal repair, e.g., under echocardiographic guidance.

To position the artificial chordae device, the device may be enclosed in or otherwise carried by a delivery device that may be used to first grasp the posterior leaflet of the mitral valve, e.g., at the area of leaflet prolapse. Once grasped, the delivery device may then puncture the body of the posterior leaflet. With the leaflet still grasped, the artificial chordae device may then be passed through the perforation and anchored to the posterior ventricular wall. To facilitate proper anchoring, the delivery device may be steerable.

Once anchored, the leaflet anchor may be deployed, e.g., to attach the anchor to the leaflet. In another embodiment, the anchor is initially partially deployed. In the partially deployed state, the length of the artificial chordae device, i.e., the distance between the leaflet anchor and the tissue anchor may be adjusted. Next, the leaflet may be released from the delivery system, and the length of the artificial chordae device may be adjusted until a point of optimal coaptation and elimination of regurgitation is identified, e.g., as visualized by echocardiography. Once in optimal position, the leaflet anchor may be fully deployed, i.e., released from the delivery system, locking the artificial chordae device at the proper length. With the device in place, the delivery device may be removed.

In accordance with an exemplary embodiment, a device is provided for percutaneous mitral repair of a mitral valve within a heart of a subject that includes a tubular member comprising a proximal portion, a distal portion sized for introduction into a patient's vasculature, and a grasper on the distal portion for grasping a posterior leaflet of a mitral valve; and an artificial chordae carried on the distal portion of the tubular member, the artificial chordae comprising one or more filaments including a distal end having a tissue anchor fixed thereto for securing the distal end to a posterior ventricular wall of the heart, and a leaflet anchor proximal to the distal end for engaging the posterior leaflet grasped by the grasper, the leaflet anchor a set distance along the one or more filaments to fix a distance between the leaflet anchor and the tissue anchor such that the artificial chordae, when released from the distal portion, improves coaptation of the mitral valve and/or reduces regurgitation.

In accordance with another embodiment, a device is provided for percutaneous mitral repair of a mitral valve within a heart of a subject that includes a tubular member comprising a proximal portion, a distal portion sized for introduction into a patient's vasculature, and a grasper on the distal portion for grasping a posterior leaflet of a mitral valve; and an artificial chordae carried on the distal portion of the tubular member, the artificial chordae comprising one or more filaments including a distal end having a tissue anchor fixed thereto for securing the distal end to a posterior ventricular wall of the heart, and a leaflet anchor proximal to the distal end for engaging the posterior leaflet grasped by the grasper such that the artificial chordae, when released from the distal portion, improves coaptation of the mitral valve and/or reduces regurgitation.

In accordance with still another embodiment, a device is provided for percutaneous mitral repair of a mitral valve within a heart of a subject that includes a tubular member including a proximal portion, a distal portion sized for introduction into a patient's vasculature, a grasper on the distal portion for grasping a posterior leaflet of a mitral valve; and an artificial chordae carried on the distal portion of the tubular member, the artificial chordae comprising one or more filaments including a distal end having a tissue anchor fixed thereto for securing the distal end to a posterior ventricular wall of the heart, and a leaflet anchor proximal to the distal end for engaging the posterior leaflet grasped by the grasper, the leaflet anchor movable along the one or more filaments to adjust a distance between the leaflet anchor and the tissue anchor until the artificial chordae is released from the distal portion to improve coaptation of the mitral valve is improved and/or eliminate regurgitation.

In accordance with another exemplary embodiment, a method is provided for mitral repair of a mitral valve within a heart of a subject that includes introducing a distal portion of a delivery device carrying an artificial chordae into the vasculature of the subject; advancing the distal portion into a right atrium of the heart and trans-septally from the right atrium into a left atrium of the heart; directing the distal portion through a mitral valve of the heart into a left ventricle of the heart; securing a distal end of the artificial chordae to a posterior ventricular wall of the heart adjacent the mitral valve; engaging a leaflet anchor on the artificial chordae to a posterior leaflet of the mitral valve; and removing the delivery device from the subject's body. Optionally, a distance between the leaflet anchor and the distal end of the artificial chordae may be adjusted and set before introduction into the patient's body, e.g., using imaging to approximate the distance. Alternatively, a distance between the leaflet anchor and the distal end of the artificial chordae may be adjusted until coaptation of the mitral valve is improved and/or regurgitation is eliminated and then fixed, e.g., using echocardiography and/or other imaging to monitor the mitral valve during the adjustment.

In one embodiment, the leaflet anchor may be fixed to the posterior leaflet such that the leaflet anchor follows movement of the posterior leaflet, e.g., during normal cardiac cycles. Alternatively, the leaflet anchor may be fixed immediately above the posterior leaflet to limit movement of the posterior leaflet away from the distal end, i.e., past the leaflet anchor, while allow the posterior leaflet to move freely along the artificial chordae along the distance between the leaflet anchor and the distal end.

In accordance with still another embodiment, a system is provided for percutaneous mitral repair of a mitral valve within a heart of a subject that includes a tubular member comprising a proximal end, a distal end sized for introduction into a patient's vasculature, and a lumen extending between the proximal and distal ends, and a delivery device comprising a proximal portion, a distal portion sized for introduction through the lumen of the tubular member, and a grasper on the distal portion for grasping a posterior leaflet of a mitral valve. An artificial chordae is carried on the distal portion of the delivery device, the artificial chordae comprising one or more filaments including a distal end having a tissue anchor for securing the distal end to a posterior ventricular wall of the heart, and a leaflet anchor proximal to the distal end for engaging the posterior leaflet grasped by the grasper, the leaflet anchor movable along the one or more filaments to adjust a distance between the leaflet anchor and the tissue anchor until the artificial chordae is deployed from the distal portion to fix the length of the artificial chordae, e.g., to improve coaptation of the mitral valve is improved and/or eliminate regurgitation.

Other aspects and features of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures:

FIG. 1 is a side view of an exemplary embodiment of an artificial chordae device.

FIG. 2 is a perspective view an exemplary delivery device for delivering and implanting the artificial chordae device shown in FIG. 1.

FIGS. 3A and 3B are side and top views, respectively, of a distal portion of the delivery device shown in FIG. 2.

FIG. 3C is a longitudinal cross-sectional side view of the delivery device shown in FIG. 2.

FIGS. 4A and 4B are details of a distal end of a delivery shaft including connectors for coupling the artificial chordae device to the delivery device, e.g., to facilitate securing a tissue anchor on the artificial chordae device to the ventricular wall.

FIG. 5 is a cross-sectional view of a heart showing an exemplary method for implanting an artificial chordae device using the delivery device of FIG. 2.

### DETAILED DESCRIPTION OF THE EXEMPLARY EMBODIMENTS

Before the exemplary embodiments are described, it is to be understood that the invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

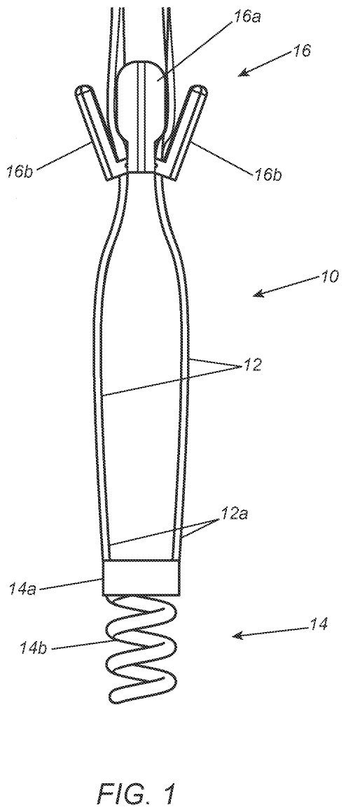

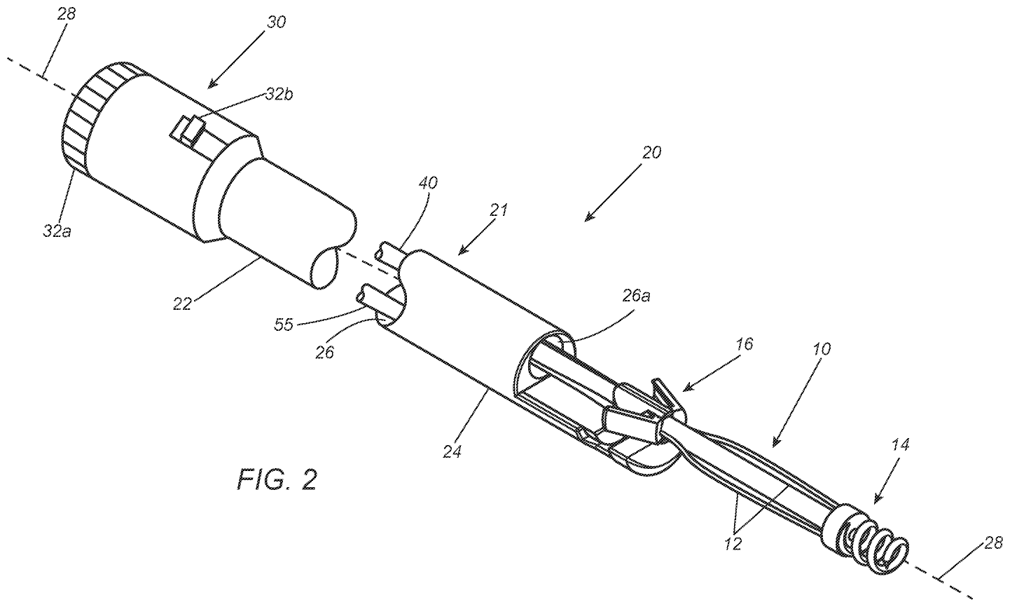

Turning to the drawings, FIG. 1 shows an exemplary embodiment of an artificial chordae device 10 that may be implanted to perform mitral repair of a mitral valve within a heart of a subject, percutaneously while the heart continues to beat. General, the artificial chordae device 10 includes one or more elongate filaments 12, a tissue anchor 14 fixed to a distal end 12a of the filament(s) 12, and a leaflet anchor 16. In one embodiment, the leaflet anchor 16 is slidable on the filament(s) 12 proximal to the tissue anchor 14. In the example shown, a pair of filaments 12 are provided with the distal end 12a of each filament 12 permanently attached to the tissue anchor 14, each filament 12 having sufficient length to extend into a delivery device (not shown, see, e.g., delivery device in FIG. 2) and/or from the subject's body. The filament(s) 12 may be formed from polytetrafluoroethylene (PTFE) or other flexible, inelastic material to provide artificial chordae, e.g., similar to conventional suture materials.

In the example shown, the tissue anchor 14 includes a ring or base member 14a, e.g., having a cylindrical or disc shape, and a helical screw 14b permanently attached to and extending distally from the base member 14a. Alternatively, other anchor elements including one or more screws, barbs, and the like may be provided that may be directed into tissue to substantially permanently anchor the tissue anchor 14 to the wall of a heart. The components of the tissue anchor 14 may be formed from rigid biocompatible materials, such as stainless steel or other metal, plastic, or composite materials, e.g., integrally molded, cast, machined, and the like, or formed separately and permanently attached together, e.g., by one or more of welding, bonding with adhesive, fusing, and the like.

The leaflet anchor 16 is configured to attach the filament 12 to a valve leaflet (not shown), i.e., setting the length of the artificial chordae device 10. In one embodiment, the distance between the leaflet anchor 16 and the tissue anchor 14 along the filament 12 is static. This distance may be selected from a range of options prior to the operation, e.g., by approximating the desired distance between a target anchor location on the posterior ventricular wall and the posterior leaflet, based on human echocardiography or other imaging. In exemplary embodiments, the leaflet anchor 16 may be a knot connected to the filament 12, a swaged or crimped anchor, e.g., made of metal or plastic, a PTFE felt anchor, or any other anchor to prevent the filament 12 from dislodging from the valve leaflet. Further alternatives for the leaflet anchor 16 are described elsewhere herein. In another embodiment, this leaflet anchor 16 may be adjusted prior to final securing to the leaflet and/or before introduction into a subject's body. In the example shown in FIG. 1, the leaflet anchor 16 includes a central member 16a and a pair of hinged members 16b that allow the filaments 12 to slide freely between them until the hinged members 16b are actuated to engage the filaments 12, i.e., to fix the leaflet anchor 16 at a desired axial location on the filaments 12 and/or preventing proximal movement of the anchor beyond a desired location of the filaments 12, thereby preventing subsequent movement away from the tissue anchor 14, as described further elsewhere herein. Optionally, the central member 16a may include a tubular body, e.g., sized to be slidably received over a delivery shaft (e.g., as described elsewhere herein). Alternatively, the central member 16a may have other shapes.

In further alternatives, the leaflet anchor 16 may include one or more locking elements that may be actuated to frictionally clamp the filament(s) 12 or otherwise secure the leaflet anchor 16 axially relative to the filament(s) 12. In another alternative, the leaflet anchor 16 may include a body including a hole or other aperture (not shown) that may slidably receive a filament 12 therethrough, but having a diameter too small to permit a knot advanced over the filament 12 from passing therethrough, thereby preventing the leaflet anchor 16 from passing over such a knot, e.g., as described elsewhere herein. An example of such a deployment mechanism of the leaflet anchor 16 onto the valve leaflet 98a is a staple or a needle (not shown) that punctures through the leaflet and is secured on the other side of the leaflet. The leaflet anchor 16 may be formed from metal, plastic, or composite materials, e.g. by molding casting, three-dimensional printing and the like.

Turning to FIG. 2, an exemplary embodiment of a delivery device 20 is shown for introducing, manipulating, and implanted the artificial chordae device 10. The artificial chordae device 10 and delivery device 20 may be part of a system or kit for performing a procedure, e.g., for percutaneous mitral repair of a mitral valve within a beating heart of a subject. For example, the system may include a guide catheter or sheath (not shown) including a proximal end, a distal end sized for introduction into a subject's body, and a guide lumen extending between the proximal and distal ends for introducing the delivery device 20 therethrough. In addition or alternatively, the system may include a cutting instrument (not shown), e.g., including an elongate shaft carrying a scissors or other cutting element on its distal end that may be sized to be introduced through the guide catheter, and/or a knot pusher (also not shown), e.g., including a lumen for receiving one or more filaments of the artificial chordae therein and a distal end sized and/or shaped to push a sliding knot on the filament(s) distally, e.g., from a proximal end of the filament(s) to a location adjacent the leaflet anchor as the knot pusher is advanced over the filament(s). Optionally, the system may also include one or more guidewires (also not shown), which may be used to introduce the guide catheter and/or the delivery device 10, a puncture device (also not shown) for creating a trans-septal opening in the septum of a heart, and the like. For example, as described further elsewhere herein, the guide catheter and/or guidewire may be introduced into a subject's vasculature, e.g., percutaneously into the subject's venous system, advanced into the right atrium of the subject's heart, a puncture device deployed to create a transseptal puncture in the atrial septum, and the guide catheter may be advanced into the left atrium of the heart to allow introduction of the delivery device 20 and/or other instruments.

With continued reference to FIG. 2, the delivery device 20 generally includes an elongate tubular member or body 21 including a proximal end or portion 22, e.g., including a handle or hub 30 sized and/or shaped to facilitate manipulation of the delivery device 20, a distal end or portion 24 sized for introduction into the subject's body, e.g., through a guide catheter (not shown), and one or more lumens 26 extending between the proximal and distal ends 22, 24, thereby defining a longitudinal axis 28 for the delivery device 20. The delivery device 20 may have sufficient length such that the distal end 24 may be introduced into a subject's heart while manipulating the handle or hub 30, e.g., from outside a percutaneous access site (not shown).

The tubular member 21 may be substantially flexible, semi-rigid, and/or rigid along its length, and may be formed from a variety of materials, including plastic, metal, and/or composite materials, as is well known to those skilled in the art. For example, in one embodiment, the tubular member 21 may have a substantially uniform construction and size along its length between the proximal and distal ends 22, 24. Alternatively, a distal region of the tubular member 21, e.g., immediately adjacent the distal end 24, may be substantially flexible to facilitate advancement through tortuous anatomy, while a proximal region, e.g., extending a predetermined distance from the proximal end 22 may be semi-rigid or rigid to enhance pushability and/or torqueability of the tubular member 21 without substantial risk of buckling or kinking. Optionally, the delivery device 20 may be steerable, e.g., including one or more steering lumens (not shown) extending between the proximal and distal ends 22, 24 for slidably receiving a wire or other steering element that may be coupled to a ring or other element (also not shown) adjacent the distal end 24. The steering element(s) may be coupled to an actuator (not shown) on the handle or hub 30, which may be actuated to bend or otherwise deflect the distal region, e.g., proximal to the distal end 24, as desired.

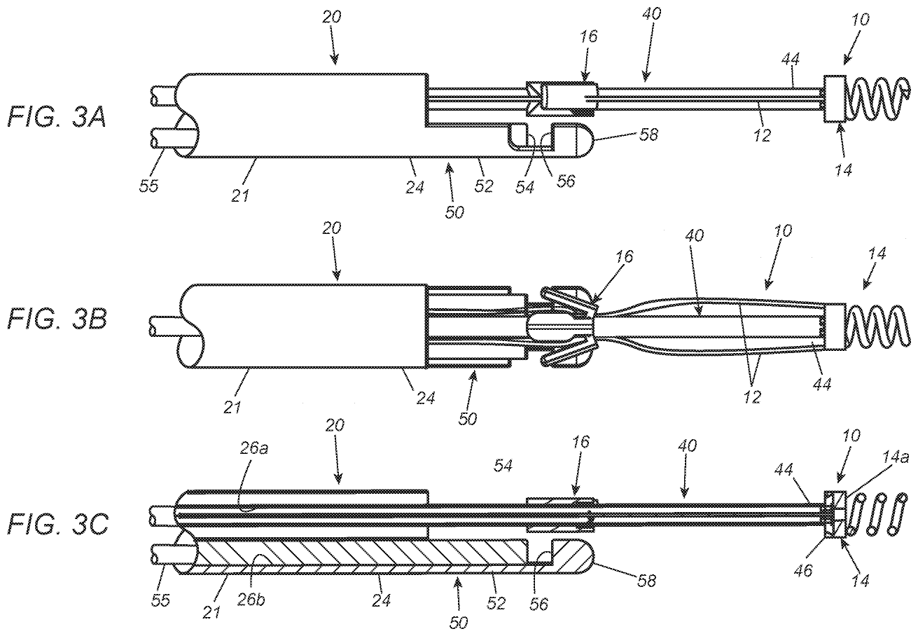

As best seen in FIGS. 3A-3C, the delivery device 20 includes a delivery shaft 40 including a shaft distal end 44 that extends from the distal end 24 of the tubular member 21 that carries the artificial chordae device 10 during introduction and implantation. The delivery shaft 40 may be slidably received within a delivery shaft lumen 26a extending between the proximal and distal ends 22, 24 such that a proximal end (not shown) of the delivery shaft 40 is coupled to an actuator 32a on the handle or hub 30. For example, as described further elsewhere herein, the actuator 32a may be rotated about the axis 28 and/or otherwise manipulated to rotate the delivery shaft 40, thereby rotating the artificial chordae device 10, e.g., for threading the screw 14b into tissue. The delivery shaft 40 may have a substantially uniform cross-section between the proximal and distal ends or may change shape and/or size within different regions of the tubular member 21, if desired.

The delivery shaft 40 may be fixed axially relative to the distal end 24. Alternatively, the delivery shaft 40 may be movable axially relative to the distal end 24, e.g., to advance and/or retract the artificial chordae device 10 relative to the distal end 24. In this alternative, the actuator 32a may also be movable axially relative to the handle or hub 30. For example, the delivery shaft lumen 26a may include a recess (not shown) adjacent the distal end 24 sized to at least partially receive the artificial chordae device 10, e.g., to retract the screw 14b proximal to a distal tip 58 of the delivery device 20.

The leaflet anchor 16 may be disposed around or otherwise adjacent the delivery shaft 40 proximal to the distal end 44, e.g., with the filaments 12 extending along the delivery shaft 40, e.g., from the distal end 44 into the delivery lumen 26a. For example, the filaments 12 may extend proximally through the delivery lumen 26a to the handle or hub 30, e.g., such that proximal ends (not shown) of the filaments 12 may be accessed and/or manipulated, e.g., to tie sliding knots that may be advanced over the filament(s) 12.

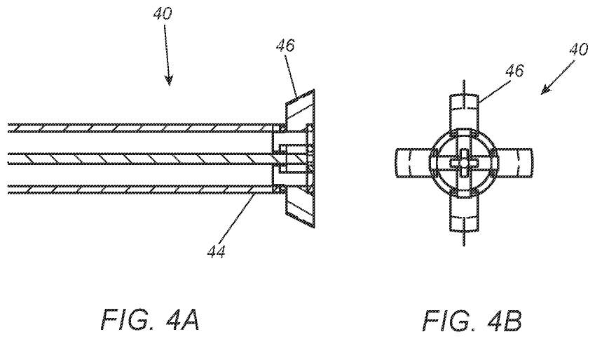

With additional reference to FIGS. 4A and 4B, the delivery shaft 40 includes one or more connectors 46 on the distal end 44, e.g., for releasably engaging the base 14a of the tissue anchor 14. For example, as shown, the connectors 46 include a plurality of radial tines that may be received in corresponding recesses or engage other features (not shown) in the base 14a, e.g., such that rotation of the delivery shaft 14 causes the base 14a and, consequently the screw 14b, to rotate. The connectors 46 may be released or otherwise disengaged from the base member 14a after securing the screw 14b in tissue, as described elsewhere herein.

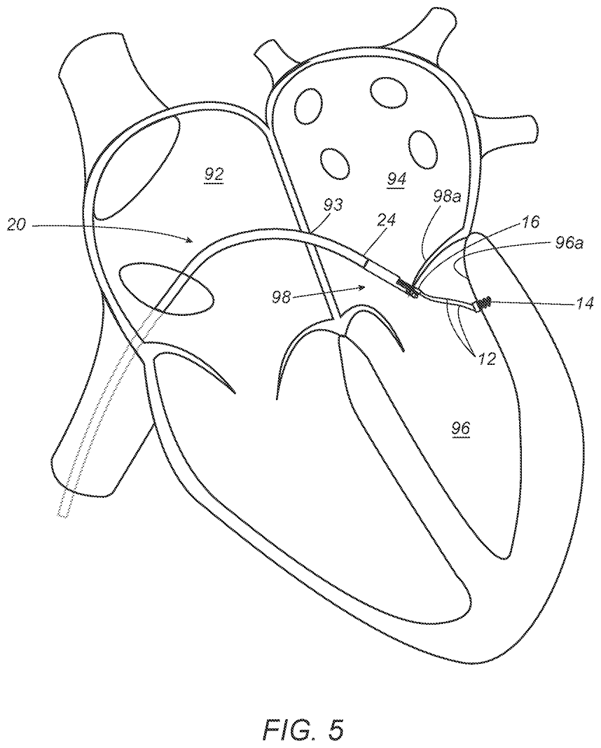

Returning to FIGS. 3A-3C, the delivery device 20 also includes a grasper mechanism 50 on the distal end 24, e.g., including a fixed jaw or extension 52 extending axially from the distal end 24 and a movable jaw 54 that may be actuated, e.g., directed axially using an actuator 32b on the handle or hub 30, to releasably grasp a posterior leaflet 98a of a mitral valve 98, e.g., as shown in FIG. 5 and described elsewhere herein. In the example shown, the fixed jaw 52 includes a recess 56 that may receive a tip of a leaflet whereupon the movable jaw 54 may be advanced distally to atraumatically grab the leaflet. The movable jaw 54 may be coupled to the actuator 32b using an elongate actuator element 55, e.g., a wire, rod, and the like, slidably received within a jaw lumen 26b that extends between the proximal and distal ends 22, 24 of the tubular member 21.

Optionally, the fixed jaw 52 may terminate in a rounded and/or other atraumatic distal tip 58, e.g., to prevent the fixed jaw 52 from skiving or otherwise damaging tissue during introduction and/or manipulation. For example, if the delivery shaft 40 is movable axially, the artificial chordae device 10 may be initially provided in a proximal or retracted position with the screw 14b adjacent the fixed jaw 52 proximal to the distal tip 58 (not shown). Once the delivery device 20 is positioned adjacent a target anchor location, the delivery shaft 40 may be advanced to a distal or active position, such as the position shown in FIGS. 3A-3C, for threading the screw 14b into tissue at the target location.

Optionally, the delivery device 20 may include one or more additional components. For example, a cutting element (not shown) may be provided on the distal end 24 adjacent the delivery lumen 26a, which may be actuated using an actuator (not shown) on the handle or hub 30 to cut, break, or otherwise sever the filament(s) 12. In addition or alternatively, a needle or other puncturing element (not shown) may be provided on the distal end 24 that may be actuated to puncture a leaflet secured by the jaws 52, 54.

Turning to FIG. 5, an exemplary method is shown for performing mitral repair of a mitral valve 98 within a heart 90 of a subject via the vasculature of the subject. For example, initially, a femoral or other peripheral vein of the subject may be accessed, e.g., using the Seldinger technique, and a guidewire (not shown) may be passed through the patient's vasculature into the right atrium 92 of the patient's heart 90, e.g., while the heart 90 continues to beat normally. A distal end of a guide catheter (also not shown) may be advanced over the guidewire into the right atrium 92, the atrial septum 93 may be perforated, and the guide catheter navigated through the atrial septum 93 into the left atrium 94 of the heart 90. In an exemplary embodiment, the guide catheter may be steerable to position the distal end against or otherwise adjacent the atrial septum 93, whereupon a puncture device (not shown) may be advanced through the guide catheter to puncture the atrial septum 93. The guide catheter may be advanced into the left atrium 93, e.g., over the puncture device, which may then be removed.

With the guide catheter in the left atrium 94, the distal end 24 of the delivery device 20 carrying the artificial chordae device 10 may be advanced through the guide catheter into the right atrium 92 of the heart 90 and trans-septally from the right atrium 92 into the left atrium 94. The guide catheter may remain within the left atrium 94, e.g., to further guide the distal end 24 of the delivery device 20, or may be removed, e.g., if the distal end 24 of the delivery device 20 is steerable.

The distal end 24 of the delivery device 20 may be directed at least partially through the mitral valve 98 into the left ventricle 96. For example, the distal end 24 may be directed partially between valve leaflets of the mitral valve 98, e.g., using the guide catheter or steering element(s) on the delivery device 20 itself, and the grasper 50 may be actuated to grasp the posterior leaflet 98a, e.g., by positioning the edge of the leaflet 98a in the recess 56 and then advancing the movable jaw 54 to secure the leaflet against the fixed jaw 52.

The posterior leaflet 98a may then be punctured, and the distal end 44 of the deliver shaft 40 carrying the artificial chordae device 10 may be directed through the puncture in the posterior leaflet 98a to position the tissue anchor 14 adjacent the posterior ventricular wall 96a of the heart 90. For example, the leaflet 98a may be punctured using the tissue anchor 14 by advancing the delivery shaft 40 relative to the distal end 24. Alternatively, a separate needle or other puncturing device (not shown) may be provided on the distal end 24 of the delivery device 20 that may be actuated independently to puncture the leaflet 98a. In another alternative, an independent needle or puncturing device may be provided that may be introduced via the guide catheter separate from the delivery device 20, which may be manipulated to puncture the leaflet 98a.

In still another alternative, the delivery shaft 40 and artificial chordae device 10 may be initially left out of the delivery device 20, e.g., if the delivery lumen 26a is sized to receive the artificial chordae device 10, and a needle may be introduced via the delivery lumen 26a and advanced out of the distal end 24 to puncture the leaflet 98a. In this alternative, the needle may then be removed and the delivery shaft 40 and artificial chordae device 10 introduced through the delivery lumen 26a out of the distal end 24 through the resulting puncture into the left ventricle 96.

With the distal end 44 of the delivery shaft 40 and the tissue anchor 14 positioned within the left ventricle 96, the tissue anchor 14 may then be secured to the posterior ventricular wall 96a adjacent the mitral valve 98 to position the tissue anchor 14 adjacent the posterior wall of the heart within the left. For example, with the screw 14b directed against the ventricular wall 96a, the actuator 32a on the handle or hub 30 may be rotated, thereby threading the screw 14b into the tissue. The entire distal end 24 may be manipulated, e.g., using the guide catheter and/or steering element(s) on the delivery device 20 to position the screw 14b against the wall 96a before threading the screw 14b. Alternatively, the delivery shaft 40 may be advanceable relative to the distal end 24, e.g., to direct the screw 14b against the wall without moving the leaflet 98a held by the grasper 50.

The leaflet anchor 16 may then be secured to the posterior leaflet 98a, e.g., while stabilized by the grasper 50. In an exemplary embodiment, deploying the leaflet anchor 16 secures the valve leaflet 98a to the leaflet anchor 16, preventing significant movement of the leaflet 98a away from the tissue anchor 14 beyond the filament 12 length. This prevents prolapse of the posterior leaflet 98a into the left atrium 94 and restores valve coaptation to reduce regurgitation. In an alternative embodiment, before securing the leaflet anchor 16, an axial location of the leaflet anchor 16 may be adjusted along the filaments 12 to adjust the distance between the anchors 14, 16, e.g., until coaptation of the mitral valve 98 is improved and/or regurgitation is eliminated, e.g., while monitoring using echocardiography or other external imaging. For example, the leaflet 98a may be released from the grasper 50 and the distal end 24 of the delivery device 20 withdrawn from the valve 98, e.g., into the left atrium 94 or removed into the guide catheter, thereby allowing the mitral valve 98 to open and close normally while the heart 90 beats. The mitral valve 98 may then be monitored using external imaging as the leaflet anchor 16 is adjusted to limit movement of the posterior valve 98a, e.g., to prevent prolapse into the left atrium 94.

In an embodiment, with the leaflet anchor 16 positioned above the leaflet 98a, i.e., within the left atrium 94, a sliding knot (not shown) may be advanced over one or more both of the filaments 12, e.g., from outside the subject's body along the entire length of the filament(s) 12, e.g., using a knot pusher (not shown) inserted into a port in the handle or hub 30 from outside the subject's body, through the delivery lumen 26a of the delivery device 20 until the knot is positioned proximal to the leaflet anchor 16. Alternatively, a knot may be provided on the filament(s) 12 proximal to the leaflet anchor 16, e.g., preloaded within the delivery lumen 26a, before the delivery device 20 is introduced, and the knot may be advanced distally over the filament(s) 12 once the artificial chordae device 10 is deployed. In a further alternative, the delivery device 20 may be removed, and the knot pusher advanced over the filament(s) 12, e.g., through the guide catheter.

Once the coaptation is improved as desired, the leaflet anchor 16 may be locked to the leaflet 98a and/or otherwise limited from proximal movement, and the artificial chordae device 10 released from the distal portion 24. For example, a sliding knot advanced over one or more filament(s) 12 may be sufficient to prevent proximal movement of the leaflet anchor 16 from a desired location, e.g., the knot may remain stationary once tightened, thereby setting a fixed length between the leaflet anchor 16 and the tissue anchor 14 that limits movement of the posterior leaflet 98a. Optionally, one or more additional knots (not shown) may be advanced over the filament(s) 12, e.g., to further tighten the knot and/or otherwise secure the leaflet anchor 16. In another alternative, one or more knots advanced over the filament(s) 12 may provide a leaflet anchor without the leaflet anchor 16, e.g., with the knot(s) positioned immediately adjacent the posterior leaflet 98a at a desired distance from the tissue anchor 14.

In addition or alternatively, other locking mechanisms may be used to secure the leaflet anchor 16. For example, a tool may be carried on the distal end 24 or may be introduced separately from the delivery device 20 that may be used to engage and lock the leaflet anchor 16, e.g., a crimping tool that crimps the central member 16a onto the filament(s) 12 or a tool that closes the hinged members 16b.

In another alternative, the leaflet anchor 16 may be adjusted and secured at a desired location on the filament(s) 12 before introduction into the subject's body. For example, based on echocardiography and/or other external imaging, a desired distance may be selected approximating the maximum distance between a target anchor anchoring location on the posterior ventricular wall 96a and the posterior leaflet 98a.

Once the leaflet anchor 16 is secured, the filament(s) 12 proximal to the leaflet anchor 16 may be severed or otherwise separated from the artificial chordae 10, and the delivery device 20 may be removed from the subject's body. For example, the delivery device 20 may be removed and a cutting member (not shown) may be introduced, e.g., via the guide catheter, and positioned within the left atrium 94 adjacent the filament(s) 12. In one embodiment, the cutting member may include an elongate shaft carrying a scissors or other cutting element that may be positioned adjacent to the leaflet anchor 16 and actuated from its proximal to cut the filament(s) 12, e.g., individually or at the same time. Alternatively, the cutting member may include a lumen such that the cutting member may be advanced over one or more both filaments 12, e.g., until the cutting element on its distal end is positioned proximal to the leaflet anchor 16, whereupon the cutting element may be actuated to sever the filament(s) 12. In a further alternative, a cutting element may be provided on the distal end 24 of the delivery 20, e.g., adjacent or within an outlet of the delivery lumen 26a, which may be actuated to sever the filament(s) 12.

Alternatively, other approaches and/or sequences may be used to introduce and implant an artificial chordae such as those described elsewhere herein. For example, in one alternative, the distal end 24 of the delivery device 20 may be initially directed through the mitral valve to secure the tissue anchor 14 to the posterior ventricular wall 96a. Then, after grasping the posterior leaflet 98a, a retractable needle integrated within the main delivery device 20 (not shown) may be used to puncture the leaflet 98a to thread the filaments 12 through the puncture. The filaments 12 may then be tied and/or secured, e.g., via a sliding knot or crimping device (not shown) and precisely adjusted to the desired neochord length, e.g., according to external imaging to reduce prolapse.

In another alternative embodiment, a transapical approach may be used to access the left ventricle 96 and introduce the delivery device 20 carrying the artificial chordae device 10, e.g., using conventional open or minimally invasive procedures to access the apex of the heart 90 and create a passage to introduce the distal end 24 of the delivery device 20 directly into the left ventricle 96. The delivery device 20 may be manipulated to secure the tissue anchor 14 to the posterior ventricular wall 96a, e.g., immediately adjacent the annulus of the mitral valve 98 or otherwise behind the posterior leaflet 98a. The leaflet anchor 16 may then be secured to the posterior leaflet 98a and the length of the filament(s) 12 adjusted until coaptation of the mitral valve 98 is improved and/or regurgitation is reduced or eliminated, whereupon the artificial chordae device 10 may be released and the delivery device 20 removed. The access site may be closed using conventional methods. In a further alternative, the leaflet anchor 16 may be secured to the leaflet 98a before securing the tissue anchor 14, if desired.

The embodiments disclosed herein may provide one or more multiple advantages over existing methods/devices, such as:

1. The devices, systems, and methods may be minimally invasive and may not require open heart surgery or cardiopulmonary bypass.

2. The devices, systems, and methods provide a repair technique, not a replacement, which preserves the patient's native valve tissue, avoids structural valve deterioration, and/or anticoagulation required for biological or mechanical prosthetic valves.

3. The relatively short length of artificial chordae may result in relatively low forces, e.g., compared to artificial chordae secured to the apex of the heart, as the artificial chordae positions the leaflet only for coaptation and does not serve a structural role, which may translate to low failure rates.

4. As opposed to existing artificial chordae devices, which require an incision and transapical (through the heart wall) approach, the devices and systems herein may be entirely percutaneous, e.g., entering the patient's venous system and approaching the mitral valve through the septum of the patient's heart.

Additional information regarding the devices and methods herein may be found in the Appendix submitted with provisional application Ser. No. 62/945,199, filed Dec. 8, 2019. The Appendix includes two articles authored by one or more of the inventors of the present application—one describing the reason why higher forces are encountered in transapical artificial chordae devices, and one draft paper underlying the biomechanical benefits of the PPVAN operation using. Further, the entire disclosures of any references identified in the Appendix are expressly incorporated by reference herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. (canceled)

2. A device for percutaneous mitral repair of a mitral valve within a heart of a subject, comprising:
a tubular member comprising a proximal portion, a distal portion sized for introduction into a patient's vasculature, and a grasper on the distal portion for grasping a posterior leaflet of a mitral valve; and
an artificial chordae carried on the distal portion of the tubular member, the artificial chordae comprising one or more filaments including a distal end having a tissue anchor fixed thereto for securing the distal end to a posterior ventricular wall of the heart, and a leaflet anchor proximal to the distal end for engaging the posterior leaflet grasped by the grasper such that the artificial chordae, when released from the distal portion, improves coaptation of the mitral valve and/or reduces regurgitation.

3. The device of claim 2, wherein the leaflet anchor is fixed a set distance along the one or more filaments from the tissue anchor.

4. The device of claim 3, wherein the leaflet anchor comprises a knot formed in the one or more filaments at the desired distance from the tissue anchor.

5. The device of claim 3, wherein the leaflet anchor comprises a PTFE felt anchor fixed to the one or more filaments at the desired distance from the tissue anchor.

6. The device of claim 2, wherein the leaflet anchor is movable along the one or more filaments to adjust a distance between the leaflet anchor and the tissue anchor before the artificial chordae is released from the distal portion.

7. The device of claim 2, wherein the leaflet anchor is movable along the one or more filaments to adjust a distance between the leaflet anchor and the tissue anchor before introducing the artificial chordae into the subject's heart.

8. The device of claim 6, wherein the leaflet anchor is securable at a desired location on the one or more filaments after the distance is adjusted.

9. The device of claim 6, wherein the leaflet anchor comprises a slidable knot formed in the one or more leaflets.

10. The device of claim 6, wherein the leaflet comprises a anchor member configured to be crimped to the one or more filaments.

11. A device for percutaneous mitral repair of a mitral valve within a heart of a subject, comprising:
a tubular member comprising a proximal portion, a distal portion sized for introduction into a patient's vasculature, and a grasper on the distal portion for grasping a posterior leaflet of a mitral valve; and
an artificial chordae carried on the distal portion of the tubular member, the artificial chordae comprising one or more filaments including a distal end having a tissue anchor fixed thereto for securing the distal end to a posterior ventricular wall of the heart, and a leaflet anchor proximal to the distal end for engaging the posterior leaflet grasped by the grasper, the leaflet anchor movable along the one or more filaments to adjust a distance between the leaflet anchor and the tissue anchor until the artificial chordae is released from the distal portion to improve coaptation of the mitral valve is improved and/or eliminate regurgitation.

12. The device of claim 2, wherein the tubular member comprises a delivery shaft including a distal end extending from the distal portion adjacent the grasper, and wherein the artificial chordae is carried on the distal end of the delivery shaft.

13. The device of claim 12, wherein the distal end of the delivery shaft includes one or more connectors for releasably engaging the tissue anchor, the delivery shaft rotatable about a longitudinal axis to rotate the distal end of the shaft for threading the tissue anchor into a posterior wall of the heart.

14. The device of claim 13, further comprising an actuator on a handle or hub coupled to the proximal portion of the tubular member, the actuator configured to rotate the distal end of the shaft.

15. The device of claim 12, wherein the delivery shaft is movable axially relative to the distal portion of the tubular member.

16. The device of claim 12, wherein the tubular member comprises a delivery lumen extending between the proximal and distal portions of the tubular member for slidably receiving the delivery shaft.

17. The device of claim 2, wherein the grasper comprises a fixed jaw extending from the distal portion to define a distal tip and a movable jaw movable relative to the fixed jaw to releasably securing the posterior leaflet.

18. The device of claim 17, further comprising an actuator on a handle or hub coupled to the movable jaw for directing the movable jaw between a first position and a second position to selectively secure the posterior leaflet to the fixed jaw.

19. The device of claim 2, wherein the artificial chordae includes two filaments, each filament including a distal end permanently attached to the tissue anchor.

20. (canceled)

21. A system for percutaneous mitral repair of a mitral valve within a heart of a subject, comprising:
a tubular guide member comprising a proximal end, a distal end sized for introduction into a patient's vasculature, and a guide lumen extending between the proximal and distal ends;
a delivery device comprising a proximal portion, a distal portion sized for introduction through the guide lumen of the guide member, and a grasper on the distal portion for grasping a posterior leaflet of a mitral valve; and
an artificial chordae carried on the distal portion of the delivery device, the artificial chordae comprising one or more filaments including a distal end having a tissue anchor fixed thereto for securing the distal end to a posterior ventricular wall of the heart, and a leaflet anchor proximal to the distal end for engaging the posterior leaflet grasped by the grasper such that the artificial chordae, when released from the distal portion, improves coaptation of the mitral valve and/or reduces regurgitation.

22-37. (canceled)

38. A method for mitral repair of a mitral valve within a heart of a subject, comprising:
introducing a distal portion of a delivery device carrying an artificial chordae into the vasculature of the subject;
advancing the distal portion into a right atrium of the heart and trans-septally from the right atrium into a left atrium of the heart;
directing the distal portion through a mitral valve of the heart into a left ventricle of the heart;
securing a distal end of the artificial chordae to a posterior ventricular wall of the heart adjacent the mitral valve;
engaging a leaflet anchor on the artificial chordae to a posterior leaflet of the mitral valve; and
removing the delivery device from the subject's body.

39-60. (canceled)
